SEQUENCE LISTING (2 sequences)

SEQ ID NO: 1 - DNA, 19 bp, artificial
  Oligonucleotide
agatcctggctaactgttc

SEQ ID NO: 2 - DNA, 19 bp, artificial
  Oligonucleotide
tacggactcaccttgcttg

(19) United States
(12) Patent Application Publication
(10) Pub. No.: US 2009/0214524 A1
(43) Pub. Date: Aug. 27, 2009

(54) Methods and compositions for regulating cell cycle checkpoints

(75) Inventors: Geert J. P. L. Kops, CG Utrecht, CA (US); Don W. Cleveland, La Jolla, CA (US)

Correspondence Address: WOLF GREENFIELD & SACKS, P.C., 600 ATLANTIC AVENUE, BOSTON, MA, 02210-2206, US

(73) Assignee: Ludwig Institute for Cancer Research, New York, NY (US)

(21) Appl. No.: 10/584,035

(22) PCT Filed: Dec. 22, 2003
(86) PCT No.: PCT/US03/41189

§ 371 Date: Aug. 11, 2008

(51) Int. Cl. A61K 39/395; C12N 5/00; A61K 31/7052; C07H 21/00; A61P 35/00

(52) U.S. Cl. 424/133.1; 435/375; 514/44 A; 424/152.1; 424/172.1; 536/24.5

(57) ABSTRACT

The invention relates to regulation of cell cycle checkpoints, and the application of such regulation in the treatment of disease, particularly cancer.

### FIELD OF THE INVENTION

The invention relates to regulation of cell cycle checkpoints, and the application of such regulation in the treatment of disease, particularly cancer.

### BACKGROUND OF THE INVENTION

Essential for the development and maintenance of any organism is the equal distribution of genetic information during each cell division. In eukaryotes, a complex signaling cascade is activated directly after nuclear envelope breakdown (NEB) that couples a sensory mechanism for kinetochore-spindle attachment to control of mitotic progression. Unattached kinetochores arrest the cell cycle until all chromosomes have made stable bi-oriented attachments. Only then is the signal silenced and is cohesion between sister chromatids resolved, resulting in the onset of anaphase (Cleveland, D. W. et al., 2003, Cell, 112:407-421). Many molecular components of the cascade in human cells have been identified, including Mad1, Mad2, the BubR1, Bub1 and Mps1 kinases, and the CENP-E kinesin (Jin, D. Y. et al., 1998, Cell, 93:81-91; Li, Y. et al., 1996, Science, 274:246-248; Chan, G. K. et al., 1999, J. Cell. Biol., 146:941-954; Taylor, S. S. et al., 1997, Cell, 89:727-735; Stucke, V. M. et al., 2002, EMBO J., 21:1723-1732; Yao, X. et al., 2000, Nat. Cell Biol., 2:484-491; Weaver, B. A. et al., 2003, J. Cell. Biol., 1 62(4):551-563). Presumably, unattached kinetochores recruit these components, activating the BubR1 kinase and a Lu signaling cascade that, through a poorly understood series of molecular events, generates an inhibitory complex that defines the ‘wait anaphase’ signal (Shah, J. V. et al., 2000, Cell, 103:997-1000). Ultimately, this sequesters Cdc20, the obligate activator of the E3 ubiquitin ligase APC/C that is responsible for targeting securin and cyclin B1 for destruction to allow sister chromatid separation and mitotic exit (Peters, J. M., 2002, Mol. Cell., 9:931-943).

The identity of the APC/C inhibitory complex is unclear. Suggestions have included oligomeric Mad2 (Fang, G., et al., 1998, Genes Dev 12:1871-1883), BubR1 (Tang, Z., et al., 2001, Dev Cell 1:227-237), synergy between Mad2 and BubR1 (Fang, G., 2002, Mol Biol Cell 13:755-766), and a complex of Bub3, BubR1, Mad2 and Cdc20 (Sudakin, V., et al., 2001, J Cell Bioi 154:925-936). All studies, however, seem to agree that the complex probably contains either Mad2 or BubR1 or both. Indeed, both of these proteins are able to inhibit APC/C-dependent ubiquitination of substrates in vitro (Fang, G., et al., 1998, Genes Dev 12:1871-1883; Tang, Z., et al., 2001, Dev Cell 1:227-237; Li, Y., et al., 1997, Proc Natl Acad Sci USA 94:12431-12436). Alternatively, either Mad2 or BubR1 or both might be direct APC/C inhibitors by acting in different pathways that respond to lack of attachment (Mad2) or lack of tension between sister centromeres (BubR1) (Skoufias, D. A., et al., 2001, Proc Natl Acad Sci USA 98:4492-4497).

Inability to prevent anaphase onset in the presence of unattached chromosomes can have dramatic consequences. During meiosis, this generally results in embryonic lethality, except for certain combinations of sex chromosomes, as well as trisomies of chromosomes 13, 18 and 21, that later cause severe birth defects (Cohen, J., 2002, Science, 296:2164-2166). In addition, chromosome loss or gain has been implicated in carcinogenesis, perhaps through loss of essential tumor suppressors or gain of oncogenes (Lengauer, C. et al., 1998, Nature, 396:643-649). Many human cancers and cancer cell lines are indeed aneuploid, but direct evidence of chromosomal loss (frequently called chromosome instability or CIN) as a driving force for malignant transformation has not yet been provided.

Although very rare (e.g. Nakagawa, H. et al., 2002, Oncol. Rep., 9:1229-1232; Shigeishi, H. et al., 2001, Oncol. Rep., 8:791-794; Reis, R. M. et al., 2001, Acta. Neuropathol. (Berl.), 101:297-304; Sato, M. et al., 2000, Jpn. J. Cancer Res., 91:504-509; Myrie, K. A. et al., 2000, Cancer Lett., 152:193-199), mutations in Bub1 and BubR1 have been found in some human cancer cell lines that display chromosome instability, and these mutations were argued to interfere with mitotic checkpoint signaling in a dominant manner (Cahill, D. P. et al., 1998, Nature, 392:300-303). A causative role for such mutations in tumorigenesis, however, was recently challenged in a study that reported a robust mitotic checkpoint response in these cell lines (Tighe, A. et al., 2001, EMBO Rep., 2:609-614). The best evidence to date for a role of impaired mitotic checkpoint signaling in carcinogenesis comes from in vivo gene knockout studies. Mice heterozygous for the Mad2 gene develop late onset papillary lung adenocarcinomas (Michel, M. L. et al., 2001, Nature, 409:355-359), while incidence of lung tumors in mice induced by the carcinogen 7,12-dimethylbenzanthracene (DMBA) was 3-fold higher in Bub3 heterozygotes than in wild type littermates (Babu, J. R. et al., 2003, J. Cell. Biol., 160:341-353). Nevertheless, the effects of mitotic checkpoint inactivation on cellular growth properties have not been reported because homozygous mutations in all genes for checkpoint proteins tested to date cause very early embryonic lethality and no cell lines have been created from them (Babu, J. R. et al., 2003, J. Cell. Biol., 160:341-353; Kalitsis, P. et al., 2000, Genes Dev., 14:2277-2282; Putkey, F. R. et al., 2002, Dev. Cell, 3:351-365; Dobles, M. et al., 2000, Cell, 101:635-645).

Although many components of the mitotic checkpoint signaling pathway have been identified, the effects of mitotic checkpoint inactivation on cellular growth properties has not been reported. The inactivation of the mitotic checkpoint has important implications for treating cancer and accordingly there is a need to understand the effect of mitotic checkpoint inactivation, particularly as new cancer therapies.

### SUMMARY OF THE INVENTION

We have used plasmid-based small interfering RNAs to eliminate expression of one or more essential mitotic checkpoint proteins, and analyzed both the short and long term effects on cell division, chromosome distribution and viability of human cancer cells. HeLa cells lacking BubR1 or Mad2 do not mitotically arrest after disrupting spindle microtubule assembly with microtubule poisons. Abrogation of the ability to detect unattached chromosomes during mitotic progression leads to premature anaphase onset and errors in chromosome segregation which within a few rounds of division eliminates viability, in part by induction of apoptosis.

The invention provides methods and compositions for treating cancer by reducing in cancer cells expression or activity of mitotic checkpoint signaling proteins to cause acute chromosome loss and subsequent loss of cancer cell viability.

According to one aspect of the invention, methods for inducing apoptosis in a cell are provided. The methods include reducing expression or activity of one or more mitotic checkpoint molecules, preferably by reducing expression by contacting the cell with a siRNA specific for the one or more mitotic checkpoint molecules or by reducing activity by contacting the cell with an antibody that binds to the mitotic checkpoint molecule. In embodiments in which an antibody is used, the antibody is selected from the group consisting of monoclonal antibodies, human antibodies, humanized antibodies, chimerized antibodies, and antigen-binding fragments thereof. In certain embodiments, the mitotic checkpoint molecule is BubR1, Mad2, Bub3 and/or CENP-E.

The activity of the mitotic checkpoint molecules also can be reduced by contacting the cell with a molecule that inhibits kinase activity of the one or more mitotic checkpoint molecules. In such methods, the mitotic checkpoint molecule preferably is BubR1.

According to another aspect of the invention, methods for treating cancer are provided. The methods include administering to a subject in need of such treatment an effective amount of an agent that reduces expression or activity of one or more mitotic checkpoint molecules, preferably by reducing expression by contacting the cell with a siRNA specific for the one or more mitotic checkpoint molecules or by reducing activity by contacting the cell with an antibody that binds to the mitotic checkpoint molecule. In embodiments in which an antibody is used, the antibody is selected from the group consisting of monoclonal antibodies, human antibodies, humanized antibodies, chimerized antibodies, and antigen-binding fragments thereof. In certain embodiments, the mitotic checkpoint molecule is BubR1, Mad2, Bub3 and/or CENP-E.

The activity of the mitotic checkpoint molecules also can be reduced by contacting the cell with a molecule that inhibits kinase activity of the one or more mitotic checkpoint molecules. In such methods, the mitotic checkpoint molecule preferably is BubR1.

In further embodiments, an anti-cancer therapy is used in combination with the agent. Preferably the anti-cancer therapy is chemotherapy; more preferably the chemotherapy is one or more microtubule poison drugs, and the chemotherapy is not co-administered with the agent.

Methods for treating a hyperproliferative cell disease are provided according to another aspect of the invention. The methods include administering to a subject in need of such treatment an effective amount of an agent that reduces expression or activity of one or more mitotic checkpoint molecules, preferably by reducing expression by contacting the cell with a siRNA specific for the one or more mitotic checkpoint molecules or by reducing activity by contacting the cell with an antibody that binds to the mitotic checkpoint molecule. In embodiments in which an antibody is used, the antibody is selected from the group consisting of monoclonal antibodies, human antibodies, humanized antibodies, chimerized antibodies, and antigen-binding fragments thereof. In certain embodiments, the mitotic checkpoint molecule is BubR1, Mad2, Bub3 and/or CENP-E.

The activity of the mitotic checkpoint molecules also can be reduced by contacting the cell with a molecule that inhibits kinase activity of the one or more mitotic checkpoint molecules. In such methods, the mitotic checkpoint molecule preferably is BubR1.

According to still another aspect of the invention, compositions are provided that include a therapeutically effective amount of a siRNA specific for a mitotic checkpoint molecule, a therapeutically effective amount of an antibody that binds to a mitotic checkpoint molecule, and/or a therapeutically effective amount of a molecule that inhibits kinase activity of a mitotic checkpoint molecule. Antibodies can be monoclonal antibodies, human antibodies, humanized antibodies, chimerized antibodies, and antigen-binding fragments thereof. In the foregoing compositions, mitotic checkpoint molecule preferably is BubR1, Mad2, Bub3 and/or CENP-E. The foregoing composition also can include a pharmaceutically acceptable carrier.

The use of the foregoing compositions and molecules in the preparation of medicaments, particularly medicaments for treatment of cancer or hyperproliferative disease, is also provided.

These and other objects of the invention will be described in further detail in connection with the detailed description of the invention.

### BRIEF DESCRIPTION OF THE DRAWINGS

FIG. 1. siRNA-Mediated Elimination of BubR1 and Mad2 Protein. A) Western blot of HeLa cells transfected with mock or BubR1 siRNA plasmid for 24 or 48 hours analyzed for BubR1 or Mad2 protein, respectively, by serial dilutions of whole cell lysates. B) Western blot of HeLa cells transfected with mock or Mad2 siRNA plasmid for 24 or 48 hours analyzed for BubR1 or Mad2 protein, respectively, by serial dilutions of whole cell lysates. C) MACS isolation of transfected HeLa cells as above, replated for 24 hours (left), or cotransfected with pH2B-EYFP (right). Cells were fixed, extracted and stained for BubR1, CENP-E and DNA (DAPI) (left), or for Mad2 and centromeres (ACA) (right). Enlarged boxes show kinetochores.

FIG. 2. Absence of Mitotic Checkpoint Response to Colcemid in BubR1- or Mad2-Deficient Cells. A) Western blot of HeLa cells transfected with the indicated siRNA plasmids, with or without colcemid for 16 hours and immunoblotted for cyclin B1, BubR1 or actin. p-BubR1; phosphorylated BubR1. B) T98G cells transfected with the indicated siRNA plasmids, with or without colcemid for 16 hours were analyzed for BrdU incorporation. S phase indicates the percentage of the cell population that is BrdU positive. C) DNA content profiles of T98G cells transfected and treated as in B. D) Stills of timelapse movies showing live cell microscopy of HeLa cells transfected as in FIG. 1 in combination with pH2B-EYFP, treated with colcemid for 5 minutes 48 hours post transfection. Asteriks indicates NEB. E) Time-lapse sequence of cells transfected with Mad2 siRNA plasmid and pH2B-EYFP. Arrows indicate the reassembled nuclear envelope.

FIG. 3. Lack of Checkpoint Response to Unattached Chromosomes in BubR1- or Mad2-Deficient Cells. A) Stills of time-lapse movies showing live cell microscopy of HeLa cells transfected as in FIG. 1 in combination with pH2B-EYFP 48 hour post transfection. B) HeLa cells transfected with the siRNA plasmids as in FIG. 1. Transfected cells were isolated by MACS, replated onto coverslips, fixed and DNA visualized with DAPI.

FIG. 4. Mitotic Checkpoint-Independent Delay of Anaphase Onset. A) graph showing timing of mitotic progression of HeLa cells transfected, treated and analyzed as in FIG. 3A (white bars) or as in FIG. 2D (black bars). First frame of NEB was set to t=0. m, mock; B, BubR1 siRNA; M2, Mad2 siRNA. B) Western blot of HeLa cells transfected with mock, BubR1 siRNA or Mad2 siRNA plasmids or a plasmid containing the siRNA sequences for both BubR1 and Mad2 (B/M2 siRNA) were isolated by MACS and whole cell lysates immunoblotted for BubR1, Mad2 or actin. C) Stills of live cell microscopy of HeLa cells expressing H2B-EYFP and B/M2 siRNA. D) Graph showing elapsed time from NEB to anaphase onset of HeLa cells expressing indicated siRNA plasmids.

FIG. 5. BubR1 Kinase Activity is Required for Mitotic Checkpoint Signaling. A) Western blot of T98G cells transfected with mock or BubR1 siRNA plasmid in combination with either empty vector or the various myc-tagged siRNA-resistant BubR1 mutants analyzed for BubR1 or the myc epitope tag (wt, wild type; ΔB, ΔBub3; αC, BubR1ΔC; KD, K795A). B), C) Graph showing percentage BrdU positivity in BubR1 mock or siRNA transfected T98G cell samples without colcemid (B), in addition to the various siRNA resistant BubR1 alleles (C). Percentage BrdU incorporation given as percentage of control (white bars). D) Stills of live cell microscopy of BubR1 siRNA HeLa cells transfected with pH2B-EYFP and the indicated siRNA resistant BubR1 alleles. Arrows indicate unaligned chromosomes during anaphase.

FIG. 6. Mitosis Without BubR1 or Mad2 Causes Acute Chromosome Loss. A) FACS analysis of DNA content of HeLa cells transfected with siRNA plasmids along with pBabe-Puro for 24 hours and grown in puromycin-containing medium for an additional 48 hours. B) Distribution of the amount of chromosomes within a G1 population of YCA-2A3 cells transfected with mock, BubR1 siRNA or Mad2 siRNA. Image is a Z-stack projection displaying all EYFP-CENP-A-containing centromeres in one plane. Number in brackets indicates amount of chromosomes.

FIG. 7. Loss of Viability by Elimination of Mitotic Checkpoint Signaling. A) Colony outgrowth assay. B) FACS analysis of HeLa cells transfected with mock, BubR1 siRNA or Mad2 siRNA plasmids along with pBabe-Puro, analyzed for DNA content (top) and morphology (bottom). 4d, 5d, 6d=4, 5 or 6 days of growth in puromycin containing medium. The extent of cell death is shown as percentage of cells with sub-2N DNA content. Bar is 50 μm. C) FACS analysis of HeLa cells transfected as in (B), analyzed for DNA content (top) and morphology (bottom) after growth in puromycin- and colcemid-containing medium for an additional 3 or 6 days, respectively. Bar is 50 μm. D) Western blot of HeLa cells either transfected with the indicated siRNA plasmids in combination with pBabe-Puro and grown in puromycin-containing medium for the indicated amount of days, or untransfected but treated with puromycin for 1 day or colcemid for 1, 2 or 3 days, immunoblotted for p85 PARP-1 protein cleavage product, caspase-3 or actin. E) Immunostaining of HeLa cells transfected and selected as in (B), for active caspase-3.

### DETAILED DESCRIPTION OF THE INVENTION

The mitotic checkpoint is activated immediately after mitotic entry to prevent anaphase onset in the presence of unattached kinetochores. Inability to elicit a checkpoint response may contribute to developmental defects and carcinogenesis by allowing unequal segregation of chromosomes. Nevertheless, the effect of acute checkpoint deficiency on somatic cell division, the maintenance of ploidy and viability is largely unknown.

We have discovered that reducing the levels of mitotic checkpoint proteins (e.g., Mad2, BubR1 or both) using siRNAs eliminates mitotic checkpoint signaling. Replacement of endogenous BubR1 with siRNA-resistant alleles reveals an absolute requirement of BubR1-Bub3 interaction and BubR1 kinase activity in the mitotic checkpoint response. While diminished activity of the checkpoint has been implicated in carcinogenesis through an increased rate of chromosome loss, checkpoint deficient cells exit mitosis with many misaligned chromosomes, rapidly generating aneuploid progeny with a chromosomal loss rate so severe as to eliminate viability by apoptosis within a few division cycles, except when cytokinesis is also inhibited.

Therefore, we have determined that eliminating the mitotic checkpoint in human cancer cells is lethal as the consequence of massive chromosome loss. These findings have implications for inhibiting proliferation of tumor cells.

More specifically, as proof of principle we have used plasmid-based small interfering RNAs (siRNAs) to eliminate expression of either or both of two essential checkpoint proteins, and analyzed both the short and long term effects on cell division, chromosome distribution and viability of human cancer cells. HeLa cells lacking BubR1 or Mad2 do not mitotically arrest after disrupting spindle microtubule assembly with microtubule poisons. Abrogation of the ability to detect unattached chromosomes during mitotic progression leads to premature anaphase onset and errors in chromosome segregation which within a few rounds of division eliminates viability, in part by induction of apoptosis.

One aspect of the invention provides methods for reducing expression or activity of one or more mitotic checkpoint molecules to induce apoptosis of cells, particularly for treating hyperproferative cell diseases and cancer. A reduction in expression of a mitotic checkpoint molecule in a preferred method may be achieved by using the technique of RNA interference (RNAi). The use of RNAi involves the use of double-stranded RNA (dsRNA) to block gene expression. (see: Sui, G, et al, 2002, Proc Natl. Acad. Sci U.S.A. 99:5515-5520). The application of RNAi strategies for reducing gene expression specifically is understood by one of ordinary skill in the art. Reduction of the activity of mitotic checkpoint molecules can be accomplished by a variety of methods, including by use of antibodies that bind to the mitotic checkpoint molecules, dominant negative mitotic checkpoint molecules, and inhibitors of enzymatic activity of the checkpoint molecules, such as kinase inhibitors to reduce kinase activity.

In one aspect of the invention, a method is provided in which siRNA molecules are used to reduce the expression of mitotic checkpoint molecules. In one embodiment, a cell is contacted with a small interfering RNA (siRNA) molecule to produce RNA interference (RNAi) that reduces expression of one or more mitotic checkpoint molecules. The siRNA molecule is directed against nucleic acids coding for the mitotic checkpoint molecule (e.g. RNA transcripts including untranslated and translated regions). In a preferred aspect of the invention the mitotic checkpoint molecule is BubR1 and/or Mad 2. In a further preferred aspect the mitotic checkpoint molecule is one or more of the following: Bub3 and CENP-E. The expression level of the targeted mitotic checkpoint molecule(s) can be determined using well known methods such as Western blotting for determining the level of protein expression and Northern blotting or RT-PCR for determining the level of mRNA transcript of the target gene, some of which are shown in the Examples below.

Another aspect of the invention provides methods of inducing apoptosis in a cell. In a preferred aspect a siRNA molecule is administered to reduce expression of a mitotic checkpoint molecule and inactivate the mitotic checkpoint pathway. In a further aspect apoptosis can be induced by administrating a dominant-negative molecule, an antibody, or a small molecule inhibitor of BubR1 or Bub 3. These apoptosis-inducing molecules, in one aspect, may be administered as a pharmaceutical composition.

Pharmaceutical compositions of the invention can be administered in combination therapy, i.e., combined with other agents. For example, the combination therapy can include a composition of the present invention with at least one anti-tumor agent which is preferably a microtubule poison, immunomodulator, immunostimulatory agent, or other conventional therapy.

In a further aspect of the invention, methods for treating a disease or disorder are provided. In one aspect a siRNA molecule is administered and expression of a mitotic checkpoint molecule inhibited. In preferred embodiments the mitotic checkpoint molecule is BubR1, Bub3, CENP-E, or Mad 2. In another aspect of the invention the administration of antisense molecules or RNAi molecules to reduce expression level and/or function level of mitotic checkpoint molecules such as BubR1, Bub3, CENP-E, or Mad 2 polypeptides can be used in the treatment of cancer. Dominant negative molecules and other inhibitors of the function of the mitotic checkpoint molecules (such as kinase inhibitors) can similarly be used.

Such disorders include cancers, such as biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma, neurosarcoma, chondrosarcoma, Ewing sarcoma, malignant fibrous histocytoma, glioma, esophageal cancer, hepatoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

In addition to cancer, the methods of the invention can be used in the treatment of hyperproliferative cell disorders and diseases. Such disorders and diseases, as is known to one of ordinary skill in the art, are characterized by excessive cell proliferation and/or rapid cell division. Hyperproliferative diseases include, but are not limited to, psoriasis (e.g., psoriasis vulgaris, pustular psoriasis, erythrodermic psoriasis and psoriatic arthritis), immunological disorders involving undesired proliferation of leukocytes, actinic keratosis, lamellar ichthyosis, benign prostatic hyperplasia, familial adenomatosis polyposis, neuro-fibromatosis, atherosclerosis, pulmonary fibrosis, arthritis, glomerulonephritis, restenosis following angioplasty or vascular surgery, hypertrophic scar formation, inflammatory bowel disease, transplantation rejection, endotoxic shock, and fungal infections. Cells of non-cancer hyperproliferative diseases typically are non-invasive.

The mitotic checkpoint molecule antibodies or antigen-binding fragments thereof can also be utilized for in vivo therapy of cancer. The mitotic checkpoint molecule antibodies or antigen-binding fragments thereof can be used with a compound which kills and/or inhibits proliferation of malignant cells or tissues. The mitotic checkpoint molecule antibody may be administered in combination with a chemotherapeutic drug to result in synergistic therapeutic effects (Baslya and Mendelsohn, 1994 Breast Cancer Res. and Treatment 29:127-138) as described in greater detail below. In view of the results provided herein, preferred chemotherapeutic drugs do not include compounds that alter microtubule assembly or dynamics, such as paclitaxel (taxol), colecmid, nocodazole and puromycin.

Also encompassed by the present invention is a method which involves using the mitotic checkpoint molecule antibodies or antigen-binding fragments thereof for prophylaxis. For example, these materials can be used to prevent or delay development or progression of cancer.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of a target gene. In one embodiment the siRNA molecule corresponds to a region selected from a cDNA target gene beginning between 50 to 100 nucleotides downstream of the start codon. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of a mitotic checkpoint molecule is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. Preferably the nucleotide encoding sequence is part of the coding sequence of one or more of the following mitotic checkpoint genes: BubR1, Mad 2, Bub3 and CENP-E. Combinations of the foregoing can be expressed from a single vector or from multiple vectors introduced into cells.

In one aspect use of the invention a vector comprising any of the nucleotide coding sequences of the invention is provided, preferably one that includes promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA.vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2/V5-GW/lacZ, pENTR/U6, pLenti6-GW/U6-laminshrna and pLenti6/BLOCK-iT-DEST (Invitrogen). These vectors and others are available from commercial suppliers.

According to an aspect of the invention, a vector comprising any of the isolated nucleic acid molecules of the invention, operably linked to a promoter to produce siRNA molecules is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques, e.g., green fluorescent protein.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the mitotic checkpoint nucleic acid molecules in expression vectors (for example to produce siRNA), as well as to transfect host cells and cell lines, for example eukaryotic, e.g., HeLa cells. Especially useful are mammalian cells such as human, mouse, hamster, pig, goat, primate, etc. In one aspect of the invention the cells may be cancer cells.

The invention, in one aspect, also permits the construction of mitotic checkpoint gene “knock-outs” or “knock-downs” in cells and in animals, providing materials for studying certain aspects of cancer. For example, a knock-out mouse (gene disruption) or a knock-down mouse (reduced gene expression by e.g., siRNA) may be constructed and examined for clinical parallels between the model and a cancer-affected mouse with downregulated expression of a mitotic checkpoint molecule.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection using Effectene (Qiagen), transfection or infection with viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a cell surface antigen, particularly those that are readily internalized. For cancer treatment, the antigen preferably is expressed on cancer cells in greater amounts than non-cancer cells, and more preferably is exclusively expressed on cancer cells.

In one aspect of the invention a method is provided for targeting a nucleic acid molecule to a cell for therapeutic use. In a preferred embodiment of the invention the nucleic acid molecule is delivered intracellularly. In a further embodiment of the invention, an antibody is used to target a nucleic acid molecule to a cell. In yet another embodiment of the invention, an antibody can be delivered alone or together with a nucleic acid molecule. In a further aspect, an antibody is delivered in combination with a delivery vehicle, such as a liposome. The antibody includes whole antibody or fragments of antibody as is described in greater detail below.

Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Antibodies that bind mitotic checkpoint molecules can be used to reduce the function of these molecules. Preferred antibodies include antibodies that inhibit mitotic arrest mediated by the mitotic checkpoint molecules. To determine inhibition, a variety of assays known to one of ordinary skill in the art can be employed. For example, the mitosis assays set forth in the Examples can be used to determine if an antibody inhibits the appropriate mitotic response. Preferably the antibody is directed against a mitotic checkpoint protein selected from BubR1, Bub3, CENP-E and Mad 2.

As used herein, the term “antibody” refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl_{q}) of the classical complement system.

The term “antigen-binding fragment” of an antibody as used herein, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

An “isolated antibody”, as used herein, is intended to refer to an antibody which is substantially free of other antibodies having different antigenic specificities (e.g., an isolated antibody that specifically binds to a mitotic checkpoint molecule, such as BubR1 or Bub3, is substantially free of antibodies that specifically bind antigens other than the mitotic checkpoint molecule). An isolated antibody that specifically binds to an epitope, isoform or variant of a mitotic checkpoint molecule may, however, have cross-reactivity to other related antigens, e.g., from other species (e.g., BubR1 or Bub3 species homologs). Moreover, an isolated antibody may be substantially free of other cellular material and/or chemicals, although it need not be. As used herein, “specific binding” refers to antibody binding to a predetermined antigen. Typically, the antibody binds with an affinity that is at least two-fold greater than its affinity for binding to a non-specific antigen (e.g., BSA, casein) other than the predetermined antigen or a closely-related antigen.

The isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment. Alternatively, the fragments are “domain antibody fragments”. Domain antibodies are the smallest binding part of an antibody (approximately 13 kDa). Examples of this technology are disclosed in U.S. Pat. Nos. 6,248,516, 6,291,158, U.S. Pat. No. 6,127,197 and EP patent 0368684.

As used herein, antibodies also include single chain antibodies (e.g., scFvs). In some embodiments, the single chain antibodies are disulfide-free antibodies having mutations e.g. in disulphide bond forming cysteine residues. The antibodies may be prepared by starting with any of a variety of methods, including administering protein, fragments of protein, cells expressing the protein or fragments thereof and the like to an animal to induce polyclonal antibodies. Such antibodies or antigen-binding fragments thereof may be used in the preparation of scFvs and disulfide-free variants thereof. The antibodies or antigen-binding fragments thereof may be used for example to identify a target protein and/or to modulate the activity of a target protein (e.g. mitotic checkpoint molecule).

Various forms of the antibody polypeptide or encoding nucleic acid can be administered and delivered to a mammalian cell (e.g., by virus or liposomes, or by any other suitable methods known in the art or later developed). The method of delivery can be modified to target certain cells, and in particular, cell surface receptor molecules or antigens present on specific cell types. Methods of targeting cells to deliver nucleic acid constructs, for intracellular expression of the antibodies (i.e., as “intrabodies”), are known in the art. In these applications, single chain antibodies are generally used, and the size of the antibody (or fragment) is kept to a minimum to facilitate translocation into the cell. The antibody polypeptide sequence can also be delivered into cells by providing a recombinant protein fused with peptide carrier molecules. These carrier molecules, which are also referred to herein as protein transduction domains (PTDs), and methods for their use, are known in the art. Examples of PTDs, though not intended to be limiting, are tat, antennapedia, and synthetic poly-arginine; nuclear localization domains also can be included in the antibody molecules. These delivery methods are known to those of skill in the art and are described in U.S. Pat. No. 6,080,724, and U.S. Pat. No. 5,783,662, the entire contents of which are hereby incorporated by reference.

The antibodies of the present invention can be polyclonal, monoclonal, or a mixture of polyclonal and monoclonal antibodies. The antibodies can be produced by a variety of techniques well known in the art. Procedures for raising polyclonal antibodies are well known and are disclosed for example in E. Harlow, et. al., editors, Antibodies: A Laboratory Manual (1988), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide, e.g., with a mitotic checkpoint protein such as BubR1 or Bub3 in the present invention. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. See; Goding (in Monoclonal Antibodies: Principles and Practice, 2d ed., pp. 60-61, Orlando, Fla., Academic Press, 1986).

The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. In particular, mouse strains that have human immunoglobulin genes inserted in the genome (and which cannot produce mouse immunoglobulins) are preferred. Examples include the HuMAb mouse strains produced by Medarex/GenPharm International, and the XenoMouse strains produced by Abgenix. Such mice produce fully human immunoglobulin molecules in response to immunization.

Dominant negative mitotic checkpoint molecules can be used to reduce the function of these molecules. Mitotic checkpoint molecules may be modified to produce a dominant-negative version of the protein. A dominant negative polypeptide is an inactive variant of a protein, which, by interacting with the cellular machinery, displaces an active protein from its interaction with the cellular machinery or competes with the active protein, thereby reducing the effect of the active protein. Modifications to a mitotic checkpoint polypeptide are typically made to the nucleic acid which encodes the mitotic checkpoint polypeptide, and can include deletions, point mutations, truncations, amino acid substitutions and additions of amino acids or non-amino acid moieties.

The end result of the expression of a dominant negative polypeptide in a cell is a reduction in function of active proteins. One of ordinary skill in the art can assess the potential for a dominant negative variant of a protein, and use standard mutagenesis techniques to create one or more dominant negative variant polypeptides. For example, one of ordinary skill in the art can modify the sequence of the cell cycle checkpoint regulatory molecules by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. In addition, numerous mutagenesis systems and kits are commercially available. The skilled artisan then can test the population of mutagenized polypeptides for diminution in a selected and/or for retention of such an activity. Other similar methods for creating and testing dominant negative variants of a protein will be apparent to one of ordinary skill in the art.

As an example of the use of dominant negative mitotic checkpoint molecules, the Examples below show that replacement of BubR1 with a kinase inactive version eliminated colcemid-mediated mitotic checkpoint arrest. It also is known that kinase-deficient BubR1 partially prevents mitotic arrest in response to nocodazole despite the presence of endogenous BubR1 (Chan, G. K. et al., 1999, J. Cell. Biol., 146:941-954; Mao, Y. et al., 2003, Cell, 114:87-98). Examples of dominant negative BubR1 molecules include BubR1 which lacks the entire kinase domain, BubR1 which lacks kinase activity by an amino acid substitution (e.g., as shown herein where the amino acid lysine at position 795 is substituted with alanine), and BubR1 which lacks its Bub3 binding region.

Modifications also embrace fusion proteins comprising all or part of the mitotic checkpoint amino acid sequence. The use of fusion proteins is a well known method to those of skill in the art. Examples of fusion proteins include but are not limited to GST, green fluorescent protein (GFP), histidine tags, and red fluorescent protein.

As used herein with respect to polypeptides, proteins or fragments thereof, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

In a further aspect of the invention a method is provided for using molecules that inhibit enzymatic function of the mitotic checkpoint molecules. In particular, as noted above, many of the mitotic checkpoint molecules are kinases. Therefore kinase inhibitors are a preferred class of compounds that can be used in the methods of invention, e.g., for inducing apoptosis and for treating cancer and hyperproliferative cell diseases. For instance, the Examples demonstrate that BubR1 kinase activity is absolutely required for checkpoint signaling. An inhibitor of BubR1 kinase activity (or the activity of other mitotic checkpoint molecules) useful in the methods of the invention is one which reduces or prevents mitotic checkpoint arrest.

Conventional treatment for cancer that can be used in conjunction with the methods of the invention may include, but is not limited to: surgical intervention, chemotherapy, radiotherapy, and adjuvant systemic therapies. As used herein, “therapeutically useful agents” include antineoplastic agents, radioiodinated compounds, toxins, other cytostatic or cytolytic drugs, and so forth. Antineoplastic therapeutics are well known and include: aminoglutethimide, azathioprine, busulfan, carmustine, cisplatin, cyclophosphamide, cyclosporine, cytarabidine, dacarbazine, dactinomycin, daunorubicin, taxol, fluorouracil, interferon-α, lomustine, mercaptopurine, mitotane, procarbazine HC1, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). It is preferred that drugs that alter microtubule assembly or dynamics, such as paclitaxel (taxol), colcemid, nocodazole and puromycin, not be used in combination with inhibitors of expression or activity of mitotic checkpoint molecules, although it remains possible that such drugs could be used as a sequential treatment with inhibitors of expression or activity of mitotic checkpoint molecules. In the latter case, for example, a cancer patient could be treated first with microtubule poison drugs, and after that course of treatment is complete, could be treated with inhibitors of expression or activity of mitotic checkpoint molecules; this order could be reversed.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000) and interferon inducible protein 10 (U.S. Pat. No. 5,994,292). A number of antiangiogenic agents currently in clinical trials are also contemplated. Agents also include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD 1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470 and Vitaxin. additional antiangiogenic agents are described by Kerbel, J. Clin. Oncol. 19(18s):45s-51s, 2001. Immunomodulators suitable for conjugation to the antibodies include interferons, and tumor necrosis factor alpha (TNFα).

The compositions of the present invention may include or be diluted into a pharmaceutically-acceptable carrier. As used herein, “pharmaceutically acceptable carrier” or “physiologically acceptable carrier” means one or more compatible solid or liquid fillers, diluents or encapsulating substances which are suitable for administration to a human or other mammal such as a primate, dog, cat, horse, cow, sheep, or goat. Such carriers include any and all salts, solvents, dispersion media, coatings, antibacterial and antifungal agents, isotonic and absorption delaying agents, and the like that are physiologically compatible. The term “carrier” denotes an organic or inorganic ingredient, natural or synthetic, with which the active ingredient is combined to facilitate the application. The carriers are capable of being co-mingled with the preparations of the present invention, and with each other, in a manner such that there is no interaction which would substantially impair the desired pharmaceutical efficacy or stability. Preferably, the carrier is suitable for oral, intranasal, intravenous, intramuscular, subcutaneous, parenteral, spinal, intradermal or epidermal administration (e.g., by injection or infusion). Suitable carriers can be found in Remington's Pharmaceutical Sciences, Mack Publishing Company, Easton, Pa. Depending on the route of administration, the active compound, e.g., antibody or siRNA may be coated in a material to protect the compound from the action of acids and other natural conditions that may inactivate the compound.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. The components of the pharmaceutical compositions also are capable of being co-mingled with the molecules of the present invention, and with each other, in a manner such that there is no interaction which would substantially impair the desired pharmaceutical efficacy. Such preparations may routinely contain salts, buffering agents, preservatives, compatible carriers, and optionally other therapeutic agents, such as supplementary immune potentiating agents including adjuvants, chemokines and cytokines. When used in medicine, the salts should be pharmaceutically acceptable, but non-pharmaceutically acceptable salts may conveniently be used to prepare pharmaceutically-acceptable salts thereof and are not excluded from the scope of the invention.

A salt retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chioroprocaine, choline, diethanolamine, ethylenediamine, procaine and the like.

The pharmaceutical preparations of the invention also may include isotonicity agents. This term is used in the art interchangeably with iso-osmotic agent, and is known as a compound which is added to the pharmaceutical preparation to increase the osmotic pressure to that of 0.9% sodium chloride solution, which is iso-osmotic with human extracellular fluids, such as plasma. Preferred isotonicity agents are sodium chloride, mannitol, sorbitol, lactose, dextrose and glycerol.

Optionally, the pharmaceutical preparations of the invention may further comprise a preservative, such as benzalkonium chloride. Suitable preservatives also include but are not limited to: chlorobutanol (0.3-0.9% W/V), parabens (0.01-5.0%), thimerosal (0.004-0.2%), benzyl alcohol (0.5-5%), phenol (0.1-1.0%), and the like.

The formulations provided herein also include those that are sterile. Sterilization processes or techniques as used herein include aseptic techniques such as one or more filtration (0.45 or 0.22 micron filters) steps.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous or non-aqueous preparation of siRNA molecule to mitotic checkpoint molecules, and/or anti-mitotic checkpoint molecule antibody, and/or small molecule inhibitors, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulations suitable for oral, subcutaneous, intravenous, intramuscular, etc. administration can be found in Remington's Pharmaceutical Sciences, Mack Publishing Co., Easton, Pa.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The therapeutics of the invention can be administered by any conventional route, including injection or by gradual infusion over time. The administration may, for example, be oral, intravenous, intraperitoneal, intramuscular, intracavity, intratumor, or transdermal. When antibodies are used therapeutically, preferred routes of administration include intravenous and by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp. 1694-1712; incorporated by reference). Those of skill in the art can readily determine the various parameters and conditions for producing antibody aerosols without resorting to undue experimentation.

The pharmaceutical preparations of the invention, when used alone or in cocktails, are administered in therapeutically effective amounts. Effective amounts are well known to those of ordinary skill in the art and are described in the literature. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

An “effective amount” is that amount of a siRNA molecule or anti-mitotic checkpoint molecule antibody that alone, or together with further doses, produces the desired response, e.g. treats a malignancy in a subject. This may involve only slowing the progression of the disease temporarily, although more preferably, it involves halting the progression of the disease permanently. This can be monitored by routine methods. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of anti-mitotic checkpoint molecule antibody, or siRNA molecules, or BubR1 kinase inhibitors, for producing the desired response in a unit of weight or volume suitable for administration to a subject. The response can, for example, be measured by determining the physiological effects of the mitotic checkpoint molecule antibody or siRNA molecules, such as regression of a tumor or decrease of disease symptoms. Other assays will be known to one of ordinary skill in the art and can be employed for measuring the level of the response.

The doses of anti-mitotic checkpoint molecule antibody or siRNA molecules, or BubR1 kinase inhibitors, administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion.

In general, doses can range from about 10 ng/kg to about 1,000 mg/kg per day, delivered in one or more portions. Based upon the composition, the dose can be delivered continuously, such as by continuous pump, or at periodic intervals. Desired time intervals of multiple doses of a particular composition can be determined without undue experimentation by one skilled in the art. Other protocols for the administration of anti-mitotic checkpoint molecule antibody or siRNA molecules will be known to one of ordinary skill in the art, in which the dose amount, schedule of administration, sites of administration, mode of administration and the like vary from the foregoing.

Dosage may be adjusted appropriately to achieve desired drug levels, locally or systemically. Generally, daily oral doses of active compounds will be from about 0.1 mg/kg per day to 30 mg/kg per day. It is expected that i.v. doses in the range of 0.01-1.00 mg/kg will be effective. In the event that the response in a subject is insufficient at such doses, even higher doses (or effective higher doses by a different, more localized delivery route) may be employed to the extent that patient tolerance permits. Continuous i.v. dosing over, for example, 24 hours or multiple doses per day also are contemplated to achieve appropriate systemic levels of compounds.

As used herein, the term “subject” is intended to include humans and non-human animals. Preferred subjects include a human patient having a cancer disorder. Other preferred subjects include subjects that are treatable with the compositions of the invention. This includes those who have or are at risk of having a cancer. Administration of the siRNA molecules, anti-mitotic checkpoint molecule antibodies, kinase inhibitors, and other compositions described herein to mammals other than humans, e.g. for testing purposes or veterinary therapeutic purposes, is carried out under substantially the same conditions as described above.

### EXAMPLES

### Experimental Procedures

Plasmids. pSUPER-BubR1 and pSUPER-Mad2 were constructed as described (Brummelkamp, T. R. et al., 2002, Science, 296:550-553) using the sequences 5′-AGATCCTGGCTAACTGTTC-3′ (SEQ ID NO:1) and 5′-TACGGACTCACCTTGCTTG -3′ (SEQ ID NO:2), respectively. Various forms of pSUPER plasmids are available commercially from OligoEngine, Inc. (Seattle, Wash.). The double siRNA plasmid pSUPERB/M2 was created by inserting a PCR fragment containing the RNA H1 promoter and the Mad2 siRNA oligo (SEQ ID NO:2) into pSUPER-BubR1 at position 117 where an AvrII site was created by site directed mutagenesis. siRNA resistant BubR1 (pcDNA3-myc-BubR1^{ΔsiRNA} was created by site-directed mutagenesis of bases 2823 (C to A) and 2826 (G to A) in pcDNA3-myc-BubR1 (a gift of S. Taylor, Harvard Medical School, Boston, Mass., U.S.A.). BubR1^{ΔBub3}, BubR1^{ΔC and BubR}1^{K795A} alleles were created by site directed mutagenesis of pcDNA3-myc-BubR1^{ΔsiRNA} by removing basepairs 1189-1257, inserting a T at position 1519 to create a premature STOP codon, or by mutating basepairs 2383-2384 to GC, respectively. Basepair numbers refer to the BubR1 nucleotide sequence, accession number NM_{—}001211. pH2B-EYFP and pH2B-ECFP were created by inserting a fragment of H2B cDNA (a gift of K. Sullivan, University of California, San Diego, Calif., U.S.A.) into modified pEYFP or pECFP (Clontech, Palo Alto, Calif.). All constructs were verified by automated sequencing.

Cell Culture and Transfections. HeLa, YCA-2A3 (HeLa cells stably expressing EYFP-CENP-A) and T98G cells were grown in DMEM supplemented with 10% Fetal Bovine Serum and 50 μg/ml pen/strep (Gibco). Colcemid (Karyo Max, Gibco/Invitrogen, Carlsbad, Calif.) was added to cells at a final concentration of 50 ng/ml, and re-added every 2 days in experiments where treatment exceeded 2 days. Transfections were done using Effectene (Qiagen, Valencia, Calif.).

Magnetic Activated Cell Sorting (MACS). Cells were transfected with pCMV-CD20 along with the various siRNA plasmids in a 1:10 ratio. Isolation of transfected cells was performed as described (Medema, R. H. et al., 2000, Nature, 404:782-787).

Antibodies and Immunoblotting. SDS-PAGE and western blotting were standard. Antibodies used in this study were: anti-BubR1 (5F9, a gift of S. Taylor, Harvard Medical School, Boston, Mass., U.S.A.), anti-CENP-E (Hpx1, Brown, K. D. et al., 1996, J. Cell. Sci., 109:961-969), anti-myc (Myc I, (Lee, M. K. et al., 1993, J. Cell Biol., 122: 1337-1350), anti-actin (N350, Amersham Biosciences, Piscataway, N.J.), anticyclin B1 (GNS1, Santa Cruz Biotechnology, Inc., Santa Cruz, Calif.) and anti-Mad2 (C19, Santa Cruz Biotechnology, Inc), anti-p85-PARP-1 (Promega, Madison Wisc.), and anti-caspase-3 (Pharmingen, San Diego, Calif.).

Immunofluorescence. Cells grown on poly-L-lysine-coated coverslips were washed once with PBS, fixed with 4% formaldehyde (Tousimis Research Corporation, Rockville, Md.) for 10 minutes, extracted with 0.5% Triton-X100 for 5 minutes and blocked in PBS containing 0.5% Tween-20 and 3% BSA (Sigma, St. Louis, Mo.) for 1 hour. Coverslips were exposed to primary antibodies diluted in blocking buffer for 1 hour, and to secondary antibodies (Jackson ImmunoResearch, West Grove, Pa.) diluted 1:200 in blocking buffer for 1 hour in the dark. After each incubation, coverslips were washed extensively with PBS/0.5% Tween-20. Finally, coverslips were submerged in PBS containing DAPI, washed once with PBS and mounted using ProLong antifade reagent Molecular Probes, Eugene, Ore.). All treatments were performed at room temperature. Dilutions: anti-Mad2 (Covance, Princeton, N.J.) 1:100, anti-BubR1 (5F9, a gift of S. Taylor, Harvard Medical School, Boston, Mass., U.S.A.): 1:1000, anti-CENP-E (Hpx1) 1:200, ACA (a gift of K. Sullivan, University of California, San Diego, Calif., U.S.A.): 1:1000, antiactive-caspase-3 (CM1, Idun Pharmaceuticals, San Diego, Calif.) 1:200.

BrdU Incorporation Assay and FACS Analysis. T98G cells were treated with 1 μM BrdU for 1 hour and analyzed by flow cytometry as described (Medema, R. H. et al., 2000, Nature, 404:782-787). For analysis of DNA content, all cells were collected, washed with PBS and fixed overnight with 70% ethanol. The next day, cells were washed with PBS and resuspended in PBS/propidium iodide/RNAseA.

Live Cell Microscopy. HeLa cells seeded on 35 mm glass-bottom dishes (MatTek Corp., Ashland, Mass.) were transfected with pH2B-EYFP and the indicated siRNA plasmids in a ratio of 1:10. 48 hours post-transfection the medium was replaced with CO₂-independent medium (Gibco) supplemented with glutamine and 10% Fetal Bovine Serum. The dish was placed in a heat-controlled stage set to 37° C. Live cell images of H2B-EYFP and brightfield (to determine NEB and nuclear envelope reformation) were taken on a Nikon Eclipse 300 inverted microscope (Nikon USA, Melville, N.Y.) using a 60XA/1.4 objective. Z-stack images were collected by a Photometrics COOLSNAP HQ camera (Roper Scientific, Tucson, Ariz.) and transferred to computer by Metamorph software (Universal Imaging Corp., Downingtown, Pa.). Time-lapse sequences were captured with exposure times of 100 ms, at 2x2 binning and with interframe intervals of 2 minutes.

Chromosome Counts. YCA-2A3 cells were grown on poly-L-lysine-coated coverslips and transfected with pH2B-ECFP along with the various siRNA plasmids in a ratio of 1:10 for 48 hours after which they were subjected to a double thymidine block. Fifteen hours after release from the block the cells were fixed in 4% formaldehyde (Tousimis Research Corporation) and mounted. Z-stack images were collected using a 100× objective.

Colony Outgrowth Assay. Cells were transfected with the indicated siRNA plasmids and pBabe-Puro in a ratio of 10:1. Twenty four hours post-transfection, cells were diluted 10-fold and grown in puromycin-containing medium (1 μg/ml) for 9 days. Cells were fixed with methanol for 30 minutes at room temperature and stained with crystal violet.

### Results

### Mitosis in the Absence of BubR1 and Mad2 Using Small Interfering RNAs

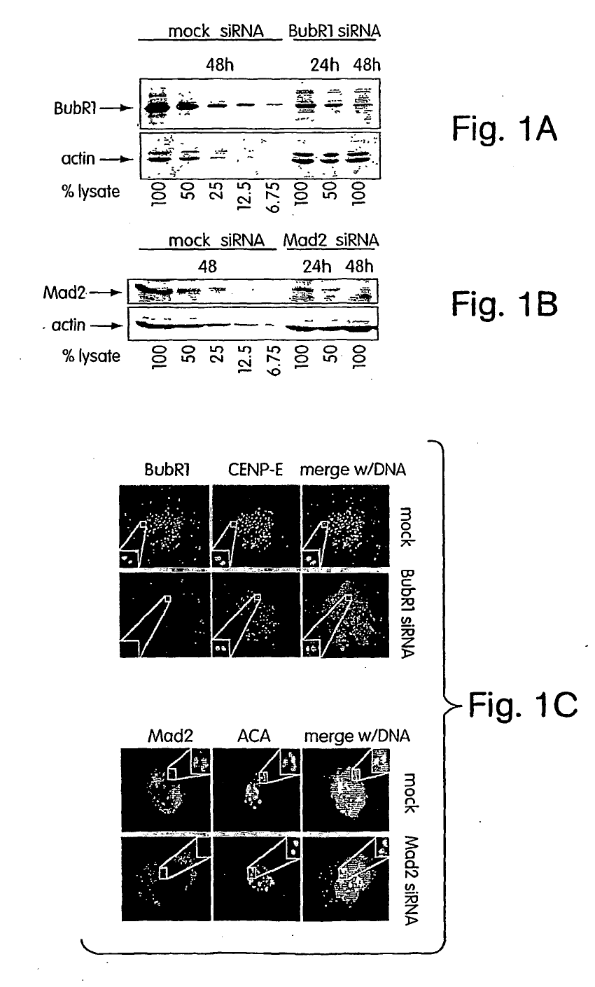

To investigate the contribution of the mitotic checkpoint to mitotic regulation and cellular viability, endogenous levels of two proposed mitotic APC/C inhibitors BubR1 and Mad2 were suppressed by plasmid-based expression of double stranded small interfering RNAs (siRNAs) (Brummelkamp, T. R. et al., Science, 296:55.0-553). Transient expression of BubR1 or Mad2 siRNA in the human cervical cancer cell line HeLa produced robust (>90%), long-term (up to 6 days) reduction in BubR1 or Mad2, respectively, as determined by immunoblotting of serially diluted HeLa cell lysates (FIGS. 1a, b). Parallel transfection of plasmids encoding a scrambled oligonucleotide sequence or one with an intentional two base mismatch had no effect on BubR1 or Mad2 levels (FIGS. 1a, b). The ˜5-10% of BubR1 and Mad2 detectable by immunoblot 48 hours post-transfection was undetectable at kinetochores, whereas CENP-E and the antigens recognized by an anti-centromere antiserum (ACA) were present at levels similar to mock transfected cells (FIG. 1c). Furthermore, protein levels of other checkpoint components including Cdc20, Mad1 and Bub1, as detected either by immunoblotting or at kinetochores, were unaffected (data not shown). This indicates that this approach can successfully produce what are essentially BubR1 and Mad2 null cells, without affecting kinetochore integrity.

### Absence of Mitotic Checkpoint Response in Cells Lacking BubR1 or Mad2

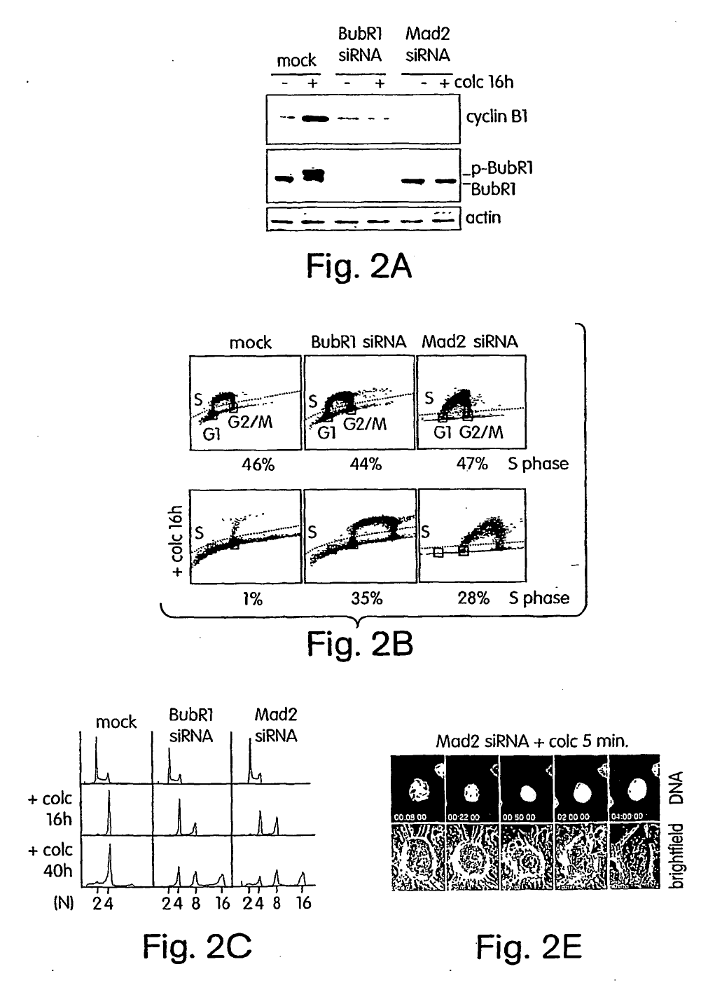

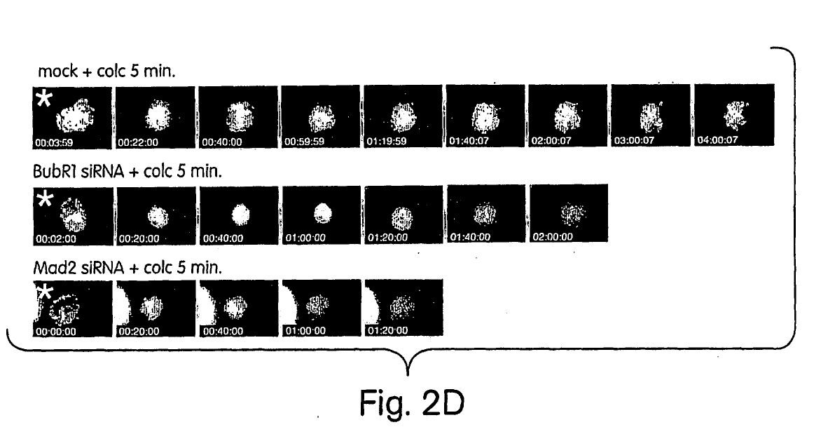

Micro-injection or electroporation of antibodies to BubR1 or Mad2 into HeLa cells has previously been shown to abolish mitotic checkpoint signaling in response to spindle disassembly induced by nocodazole (Li, Y. et al., 1996, Science, 274:246-248; Chan, G. K. et al., 1999, J. Cell. Biol., 146:941-954). It is, however, impossible with such antibody approaches to verify the specificity of antibody inhibition following acute introduction of highly concentrated antibodies or to distinguish whether the phenotype is from loss of Mad2 or BubR1 function or steric blockage of function of components bound to these proteins. To determine whether BubR1 and Mad2 are essential for mitotic checkpoint signaling, HeLa cells expressing the siRNAs were treated with the microtubule destabilizing drug colcemid. Following 16 hours of colcemid treatment, cells transfected with the mock siRNA plasmids accumulated in mitosis with 4N DNA content and high levels of cyclin B1 and phospho-BubR1 (FIG. 2a). After siRNA-mediated depletion of BubR1 or Mad2, cells did not show a mitotic arrest by any measure. BrdU incorporation experiments revealed that, despite the presence of colcemid, cells lacking BubR1 or Mad2 exited mitosis without cytokinesis and reduplicated their DNA in the subsequent S phase, yielding a significant proportion of octaploid cells after 16 hours and cells with 16N DNA content after an additional 24 hours of colcemid treatment (FIG. 2b, c).

Indistinguishable results were obtained when nocodazole or taxol was used to disrupt microtubule assembly or dynamics (data not shown). This was not just the outcome of the cell line initially chosen because it yields a very high transfection efficiency. Similar results were obtained with HeLa cells expressing EYFP-tagged Histone 2B (H2B-EYFP).

Following introduction of BubR1 and Mad2 siRNAs and addition of colcemid, mitotic HeLa cells were filmed at 2 minute time intervals to produce time-lapse movies beginning at the earliest steps of nuclear envelope disassembly. Cells expressing the mock siRNAs entered mitosis normally, but remained arrested at prometaphase by the mitotic checkpoint for at least 4 hours (the longest time point filmed) (FIG. 2d). Checkpoint-deficient cells, however, ultimately escaped mitotic arrest in the absence of sister chromatid separation and cytokinesis as visualized by chromosome decondensation and nuclear envelope reformation (FIG. 2d, e).

### Premature Anaphase in BubR1- and Mad2-Deficient Cells

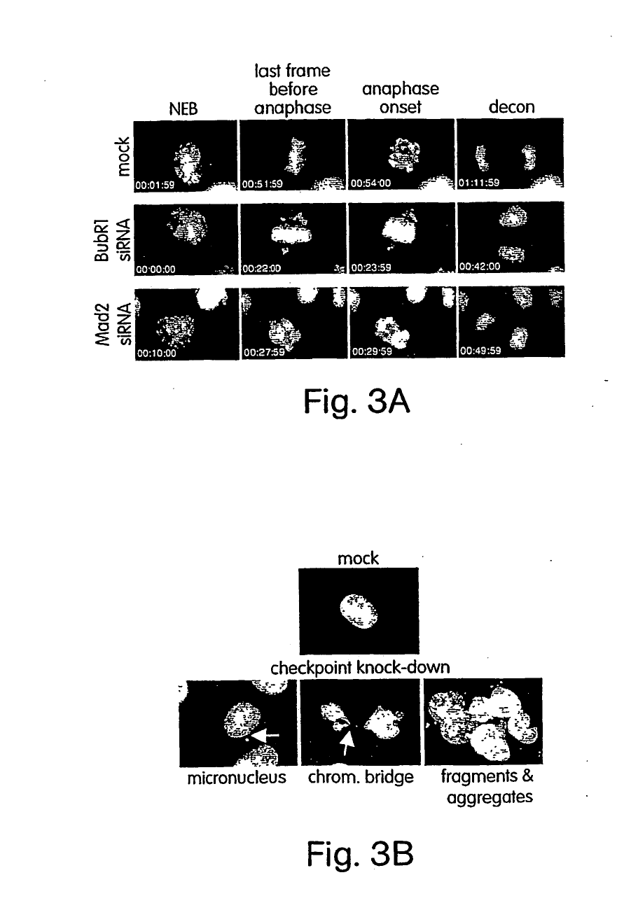

Independent of microtubule poisons, mammalian cells prevent single chromosome loss and thus guard against aneuploidy by activation of mitotic checkpoint signaling at every prometaphase, silencing it only after all kinetochores have attached to the spindle (Cleveland, D. W. et al., 2003, Cell, 112:407-421). To examine how absence of BubR1 or Mad2 affects pre-anaphase events, mitotic HeLa cells expressing H2B-EYFP with the various siRNAs were filmed in the absence of colcemid. In control cells all chromosomes were aligned approximately 18 minutes prior to onset of anaphase (FIG. 3a). In contrast, cells lacking BubR1 or Mad2 entered anaphase with many unaligned chromosomes (FIG. 3a). By 72 hours post-transfection, many obviously abnormal nuclei were present in the BubR1 or Mad2 siRNA cells, such as ones containing chromosomal bridges, micronuclei, and aggregates of malformed nuclei and nuclear fragments (FIG. 3b). Thus, BubR1 and Mad2 are each essential for the timing of normal mitosis and for the ability in such mitoses of arresting advance to anaphase until all chromosomes have attached.

### Mitotic Checkpoint-Independent Delay in Anaphase Onset

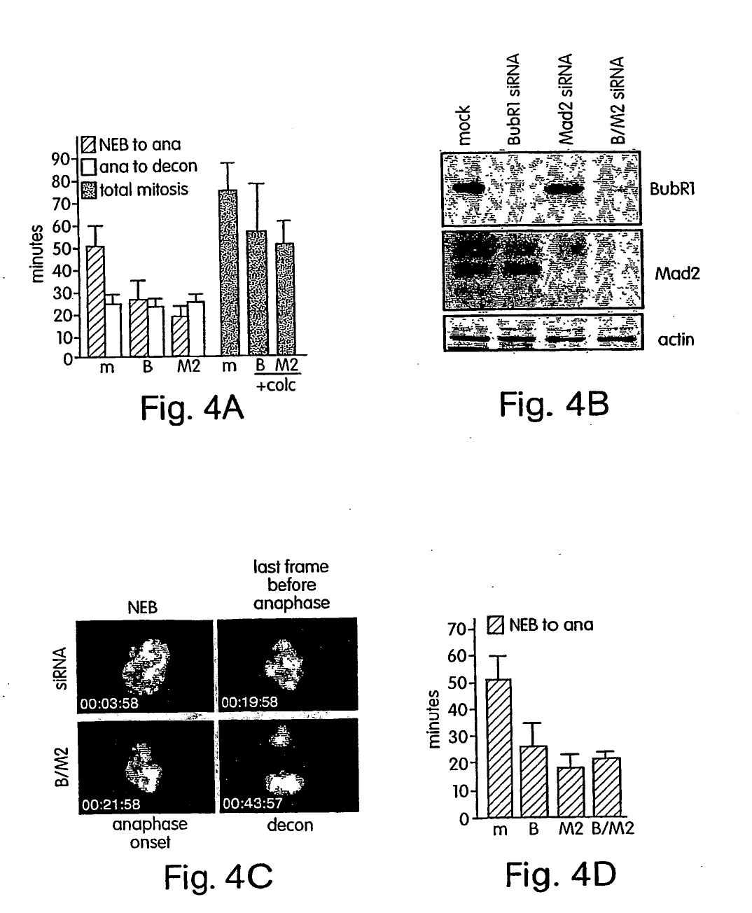

Detailed analysis of the live cell microscopy experiments revealed that BubR1- or Mad2-deficient cells required 26.0±8.6 (n=12) and 18.1±4.4 (n=14) minutes, respectively, from nuclear envelope disassembly to initiation of chromosome separation, as compared to 50.8±8.7 (n=10) minutes for control cells FIG. 4a). This delay in anaphase entry could be due to partial inhibition of APC/C, since cells depleted for BubR1 still express Mad2 and vice versa, and in both situations an inhibitory complex of APC/C containing either Mad2 or BubR1, albeit weakened, could still be an active inhibitor. Conversely, the delay could simply reflect the minimum time required for APC/C-dependent destruction of securin, activation of separase and cleavage of cohesins. To distinguish between these, siRNAs to both BubR1 and Mad2 were introduced on a single plasmid construct, resulting in an almost complete reduction in endogenous BubR1 and Mad2 (FIG. 4b). Microscopy of live cells lacking both BubR1 and Mad2 was very similar to each individual knock-down, yielding many unaligned chromosomes when anaphase ensued (FIG. 4c). Time from nuclear envelope disassembly to anaphase onset remained 21.4±3.6 (n=11) minutes, indicating no additional attenuation of APC/C inhibition (FIG. 4d). This strongly argues that absence of either APC/C inhibitor results in complete elimination of cellular mitotic APC/C inhibitory capacity, consistent with proposed models that suggest an inhibitory complex containing both Mad2 and BubR1 (Fang, G., 2002, Mol. Biol. Cell., 13:755-766; Sudakin, V. et al., 2001, Cell. Biol., 154:925-936). Moreover, from the moment of checkpoint inactivation, approximately 20 minutes are required apparently to achieve securin degradation, separase activation and cohesin cleavage, during which time some chromosomes are still able to attach to spindle microtubules and begin alignment.

### The BubR1 Kinase is Required for Checkpoint Function

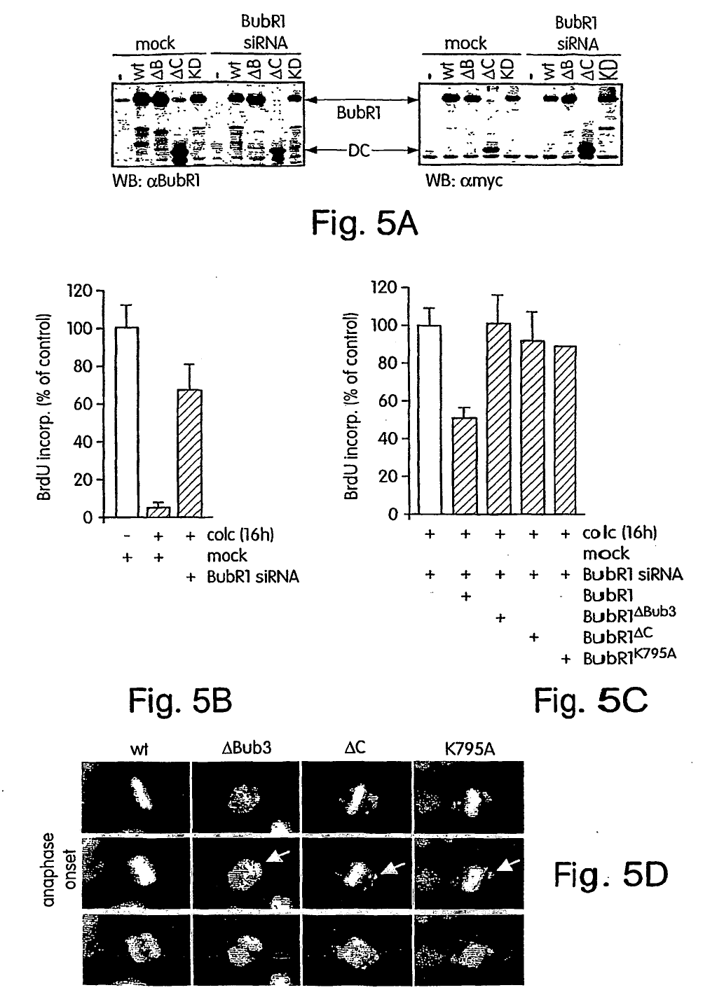

The BubR1 protein is composed of an amino-terminal Bub3-binding region required for kinetochore binding (Taylor, S. S. et al., 1998, J. Cell. Biol., 142: 1-11) and a carboxy-terminal kinase domain that is activated by binding to CENP-E (Weaver, B. A. et al., 2003, J. Cell Biol., 162(4):551-563; Mao, Y. et al., 2003, Cell, 114:87-98). The kinase activity is essential for checkpoint arrest in Xenopus laevis egg extracts, while overexpression of a kinase-deficient BubR1 partially prevents mitotic arrest in response to nocodazole despite the presence of endogenous BubR1 (Chan, G. K. et al., 1999, J. Cell. Biol., 146:941-954; Mao, Y. et al., 2003, Cell, 114:87-98). To test conclusively the requirement of BubR1 kinase in the mammalian checkpoint, siRNA transfection was used to reduce endogenous BubR1 and simultaneously express a BubR1 protein from an siRNA-resistant allele (2 bp mutation, leaving the encoded amino acids unaffected except for an amino-terminal myc epitope tag). Immunoblotting showed that exogenous BubR1 accumulated to 5-10 times the endogenous BubR1 level, and was unaffected by the siRNA that eliminated the endogenous BubR1 (FIG. 5a). BrdU incorporation coupled with filming of mitotic progression by live cell microscopy demonstrated that replacement of BubR1 with the kinase inactive version eliminated colcemid mediated mitotic checkpoint arrest. Expression of siRNA-resistant wild type BubR1 restored the checkpoint in the presence of colcemid, as seen by the 50% decrease in BrdU incorporation compared to cells depleted of endogenous BubR1 (FIGS. 5b, c). That checkpoint restoration is not complete may reflect limited accumulation of the exogenous wild type BubR1 in some cells or, alternatively, the robustness of checkpoint signaling is sensitive to the levels of BubR1 and only a subset of cells accumulate an optimal amount. Suppression of endogenous BubR1 along with expression of siRNA-resistant BubR1 lacking either the entire kinase domain (BubR1^{ΔC}), kinase activity (BubR1^{K795A}), or the Bub3 binding region (BubR1^{ΔBub3}) did not restore the checkpoint (FIG. 5c), although the accumulated protein levels were comparable to wild type exogenous BubR1 (FIG. 5a). Live cell microscopy corroborated these results: siRNA-resistant wild type BubR1 blocked anaphase entry with misaligned chromosomes in cells depleted for endogenous BubR1, whereas none of the three mutants did (FIG. 5d). In all, these data indicate that both the kinase activity and the BubR1-Bub3 interaction are essential for sustained checkpoint signaling.

### Absence of Mitotic Checkpoint Signaling Causes Massive Chromosome Loss

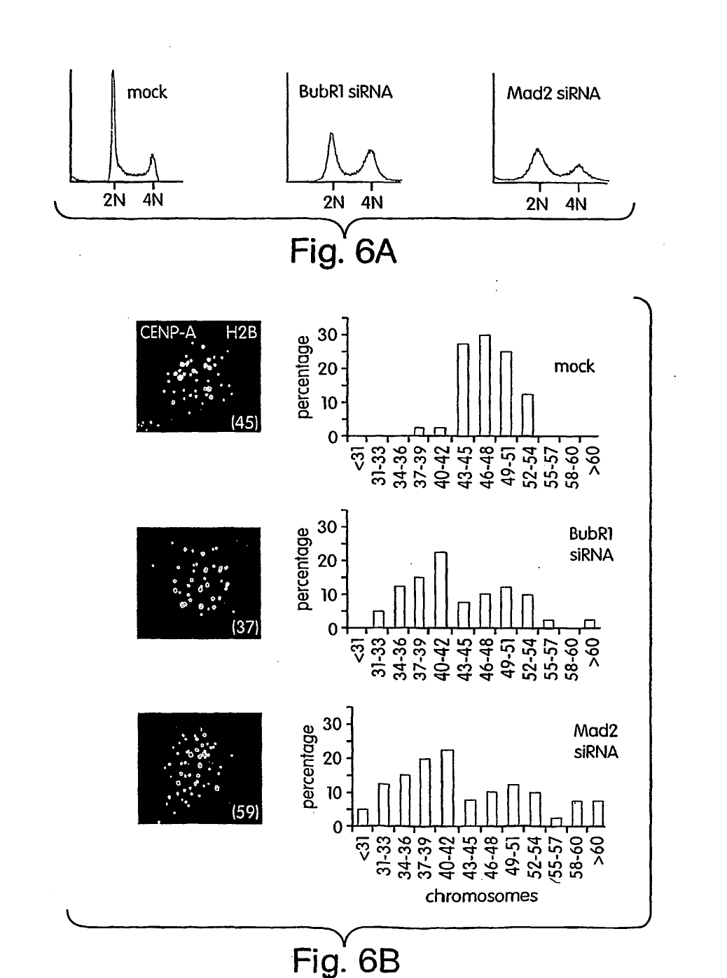

Gain and loss of chromosomes is a logical consequence of anaphase onset in the presence of misaligned chromosomes. Several studies have shown that partial inactivation of mammalian checkpoint signaling by reduction in Mad2 (Michel, M. L. et al., 2001, Nature, 409:355-359), Bub3 (Babu, J. R. et al., 2003, J. Cell Biol., 160:341-353) or CENP-E (Weaver, B. A. et al., 2003, J. Cell Biol., 162(4):551-563; Putkey, F. R. et al., 2002, Dev. Cell, 3:351-365) can indeed result in chromosome separation with partially congressed and monopolar chromosomes, as seen in this study using HeLa cells depleted for BubR1 or Mad2 (FIG. 3a). To test the consequence of acute, complete inactivation of checkpoint signaling on maintenance of ploidy, FACS profiles of HeLa cell DNA content were obtained 72 hours after transfection of BubR1 or Mad2 siRNA. This revealed that a significant proportion of BubR1 and Mad2 depleted cells had DNA contents that diverged considerably from the major 2N and 4N peaks observed in control cells, indicative of significant gain and loss of DNA within 2-3 divisions (FIG. 6a). Further, the cell population was enriched for cells in G1 by a 15 hour release from a double thymidine block and by projecting Z-stack images of nuclei of individual HeLa cells stably expressing EYFP-CENP-A (YCA-2A3 cells) all centromeres in a single cell were visualized in one plane. Most mock transfected cells had a range of 44-50 chromosomes, although deviations from these numbers were occasionally seen (FIG. 6b). Cells lacking BubR1 or Mad2, however, displayed a significantly broader range of chromosome numbers (FIG. 6b), demonstrating severe chromosome loss within one or two divisions in the absence of a functional mitotic checkpoint.

### Lethality to Mitotic Checkpoint-Deficient Cells due to Chromosome Loss

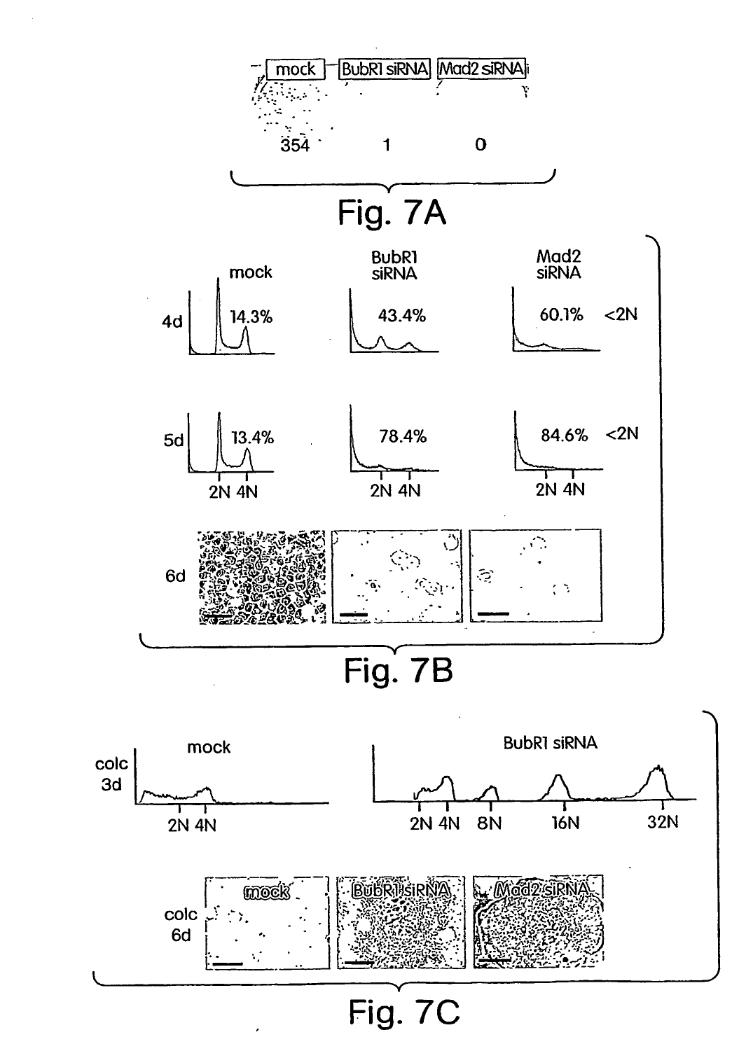

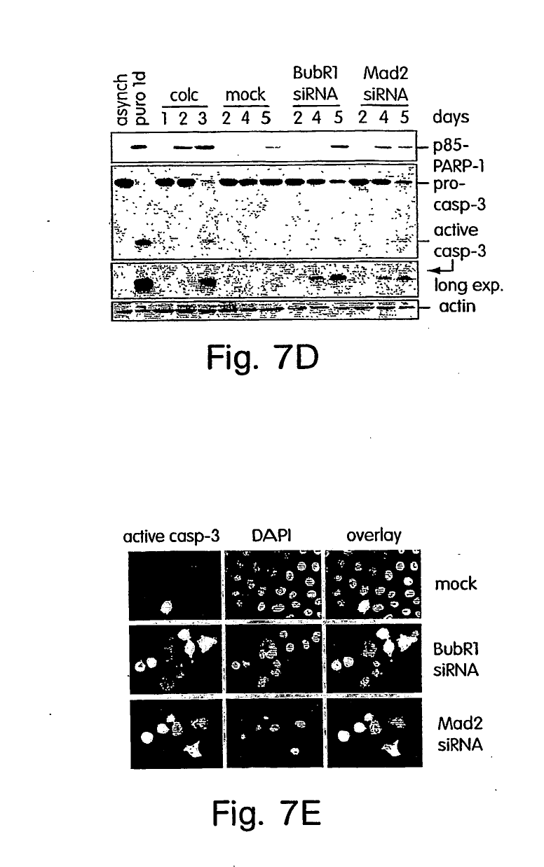

Colony outgrowth assays of cells depleted of either BubR1 or Mad2 were performed to determine whether loss of the mitotic checkpoint affected cell viability. siRNA encoding plasmids were introduced together with a plasmid carrying a puromycin resistance gene, and non-transfected cells were removed from the experiment by continuous growth in puromycin-containing medium. After 9 days surviving cells were stained with crystal violet and colonies were counted. This revealed that BubR1- or Mad2-depleted cells could form no colonies (FIG. 7a). FACS analysis further showed a large increase in the proportion of cells containing less than a 2N amount of DNA, beginning as early as 4 days post-transfection (FIG. 7b). By 6 days post-transfection, no BubR1 or Mad2 siRNA cells were viable. Since in control cells puromycin-related death occurred within the first 1-2 days of selection, death observed at 4-5 days in cells expressing BubR1 or Mad2 siRNA must reflect the absence of mitotic checkpoint signaling. Similar results were obtained with the glioblastoma cell line T98G and the osteosarcoma cell line U20S (data not shown).

Paradoxically, despite a defective mitotic checkpoint, cell death in BubR1- or Mad2-depleted cells was averted when the cells were grown chronically in the presence of colcemid (FIG. 7c). Here, despite absence of chromosome segregation and cytokinesis, giant cells and nuclei were produced as a consequence of continued cycling (FIG. 7c). Thus, loss of viability in checkpoint-deficient cycling cells was not due to activation of a death mechanism following an aberrant mitosis or by escape from such a mitosis. Rather, loss of viability probably arose directly from rapid loss of genes required for survival of individual cells. Conversely, death of control cells after chronic colcemid treatment, as measured by the proportion of cells with sub-2N DNA content, was nearly eliminated by reducing the expression of BubR1 or Mad2 (FIG. 7c).

By measuring the activation of caspase-3 as well as the appearance of the p85 cleavage product of caspase-3-cleaved poly-(ADP-ribose) polymerase-1 (PARP-1), death by prolonged colcemid treatment and death by loss of chromosomes in BubR1 or Mad2 deficient cells was shown to arise at least in part from activation of apoptotic pathways (FIG. 7d). Caspase-3 activation was already seen in the majority of cells lacking BubR1 or Mad2 after two to three divisions (FIG. 7e).

### Discussion

Through elimination of endogenous BubR1 and Mad2 protein we have shown that a) cells lose the ability to mitotically arrest in the presence of unattached chromosomes; b) BubR1 kinase activity is absolutely required for checkpoint signaling; c) a minimal period of ˜20 minutes is required for resolving sister chromatid cohesion, and d) cells lose chromosomes at a very high rate, which leads to loss of viability ultimately through programmed cell death.

Explaining the Time from Attachment of the Last Chromosome to Anaphase Onset

Timing of mitosis in PtK1 cells revealed that the time from attachment of the last chromosome, which represents the end of generation of the wait-anaphase signal, to anaphase onset is ˜23 minutes (Rieder, C. L. et al., 1994, J. Cell Biol., 127:1301-1310). Several explanations have been given for this ‘delay’, including decay of the APC/C-inhibitory activity that after falling below a certain threshold level results in acute securin destruction and subsequent synchronous chromosome segregation. In the present study, however, cells lacking BubR1 and Mad2, the only two APC/C inhibitors known to act after NEB, likely cannot assemble any such APC/C-inhibitory activity to begin with, yet a ˜20 minute time window to anaphase onset still exists (FIG. 4d). This suggests that the APC/C is activated towards securin immediately after silencing mitotic checkpoint signaling through attachment of the last chromosome (or right after NEB in the BubR1/Mad2 double siRNA cells) but ˜20 minutes are required to degrade securin, activate separase and cleave the cohesins. This view is supported by the demonstration that in cells expressing dominant-negative mBub1 a decline in levels of a securin-YFP fusion protein initiates immediately after mitotic entry and continues for ˜22 minutes before anaphase ensues (Hagting, A. et al., 2002, J. Cell Biol., 157:1125-1137). Similarly, cleavage of the cohesin subunit SCC1 in HeLa cells is apparently a gradual process (Waizenegger, I. C. et al., 2000, Cell, 103:399-410).

### BubR1 Kinase Activity in the Mitotic Checkpoint

Unlike Saccharomyces cerevisiae Mad3, BubR1 in higher eukaryotes has evolved to include a kinase domain and depletion and add-back studies in Xenopis laevis egg extracts have shown that the BubR1 kinase is indispensible for proper functioning of the in vitro checkpoint (Mao, Y. et al., 2003, Cell, 114:87-98). While human BubRl kinase activity is not required for its ability to inhibit the APC/C in vitro (Tang, Z. et al., 2001, Dev. Cell, 1:227-237), we have now shown that restoration of checkpoint signaling in BubR1-depleted cells requires BubRl kinase activity. The inclusion of a kinase domain during evolution may reflect co-evolution with its binding partner CENPE, whose binding to BubR1 stimulates the essential kinase activity (Weaver, B. A. et al., J. Cell Biol., 162 (4):551-563; Mao, Y. et al., 2003, Cell, 114:87-98). Candidate substrates known to be phosphorylated by BubR1 in vitro include Cdc20 (Wu, H. et al., 2000, Oncogene, 19:4557-4562) and the adenomatous polyposis coli (APC) gene product (Kaplan, K. B. et al., 2001, Nat. Cell Biol., 3:429-432), but the in vivo substrates and their roles in checkpoint signaling remain to be determined.

### Checkpoint Signaling, Aneuploidy and Carcinogenesis

Many solid tumors, including 85% of colon cancers, are aneuploid and have a high frequency of chromosome loss. It has been hypothesized that this is one of the driving forces of carcinogenesis in certain types of tumors through loss of essential tumor suppressor genes or by gaining copies of proto-oncogenes (Lengauer, C. et al., 1998, Nature, 396:643-649). Indeed, it seems likely that inactivating mutations in genes that guard against aneuploidy exist in cancers that exhibit chromosomal instability. This would include weakening mitotic checkpoint signaling through heterozygous loss of mitotic checkpoint genes, which has been seen to yield an increased frequency of late onset, benign lung tumors (Michel, M. L. et al., 2001, Nature, 409:355-359) or a 3 fold increase in chemically-induced tumors (Babu, J. R. et al., 2003, J. Cell Biol., 160:341-353). By eliminating either BubR1 or Mad2, we have now established that while a weakened checkpoint may enhance aspects of tumorigenesis, further silencing of it is invariably lethal to tumor cells within two to three divisions.

### Killing Cancer Cells: Targeting the Mitotic Checkpoint

Drugs that alter microtubule assembly or dynamics, especially paclitaxel (taxol), are used clinically for treatment of several human cancers. Although the mechanism of antitumorigenesis is not firmly established, concentrations of taxol that induce prolonged mitotic arrest eventually cause cell death by apoptosis (Jordan, M. A. et al., 1996, Cancer Res., 56:816-825; Jordan, M. A. et al., 1993, Proc. Natl. Acad. Sci. USA, 90:9552-9556). Similar cell death was seen here with prolonged microtubule disassembly, but death was averted when the mitotic checkpoint was inactivated (FIG. 7c). On the other hand, we now show that mitotic checkpoint inactivation also causes lethality by apoptosis through massive loss of chromosomes, but importantly only in the absence of microtubule poisons. All this suggests an intricate link between checkpoint signaling and cell death, as first suggested by Taylor and McKeon (Taylor, S. S. et al., 1997, Cell, 89:727-735).

One possibility of how drugs like taxol activate the cell death machinery is that apoptosis by prolonged mitotic arrest is indirect and due to chronically active mitotic kinases yielding hyper-phosphorylation of bcl-2 which abrogates its anti-apoptotic function (Blagosklonny, M. V. et al., 1999, Int. J. Cancer, 83:151-156; Haldar, S. et al., 1996, Cancer Res., 56:1253-1255). Although cyclin B-cdk1 has been implicated in the phosphorylation of bcl-2, the latter appears to be a poor in vitro substrate for the kinase complex (Scatena, C. D. et al., 1998, J. Biol. Chem., 273:30777-30784). On the other hand, chronic mitotic checkpoint signaling could directly modify the cell death machinery. Continued absence of attachment results in gradual accumulation of checkpoint proteins like Mad2 at kinetochores in PtK1 cells (Hoffman, D. B. et al., 2001, Mol. Biol. Cell, 12:1995-2009). Conceivably, a threshold level of active checkpoint kinase molecules might be reached that results in, for example, sufficient amounts of phosphorylated bcl-2. Although BubR1 did not increase at PtK1 kinetochores during such continued absence of attachment, other checkpoint kinases were not examined (Hoffman, D. B. et al., 2001, Mol. Biol. Cell, 12:1995-2009).

### References

1. Cleveland, D. W., Mao, Y. and Sullivan, K. F. (2003). Centromeres and kinetochores. From epigenetics to mitotic checkpoint signaling. Cell 112, 407-421.
2. Jin, D. Y., Spencer, F. and Jeang, K. T. (1998). Human T cell leukemia virus type 1 oncoprotein Tax targets the human mitotic checkpoint protein MAD1. Cell 93, 81-91.
3. Li, Y. and Benezra, R. (1996). Identification of a human mitotic checkpoint gene: hsMAD2. Science 274, 246-248.
4. Chan, G. K., Jablonski, S. A., Sudakin, V., Hittle, J. C. and Yen, T. J. (1999). Human BUBR1 is a mitotic checkpoint kinase that monitors CENP-E finctions at kinetochores and binds the cyclosome/APC. J Cell Biol 146, 941-954.
5. Taylor, S. S. and McKeon, F. (1997). Kinetochore localization of murine Bub1 is required for normal mitotic timing and checkpoint response to spindle damage. Cell 89, 727-735.
6. Stucke, V. M., Sillje, H. H., Arnaud, L. and Nigg, E. A. (2002). Human Mps1 kinase is required for the spindle assembly checkpoint but not for centrosome duplication. Embo J 21, 1723-1732.
7. Yao, X., Abrieu, A., Zheng, Y., Sullivan, K. F. and Cleveland, D. W. (2000). CENP-E forms a link between attachment of spindle microtubules to kinetochores and the mitotic checkpoint. Nat Cell Biol 2, 484-491.
8. Weaver, B. A., Bonday, Z. Q., Putkey, F. R., Kops, G. J., Silk, A. D. and Cleveland, D. W. (2003). Centromere-associated protein-E is essential for the manimalian mitotic checkpoint to prevent aneuploidy due to single chromosome loss. J Cell Biol 162(4), 551-563.
9. Shah, J. V. and Cleveland, D. W. (2000). Waiting for anaphase: Mad2 and the spindle assembly checkpoint. Cell 103, 997-1000.
10. Peters, J. M. (2002). The anaphase-promoting complex: proteolysis in mitosis and beyond. Mol Cell 9, 931-943.
11. Fang, G., Yu, H. and Kirschner, M. W. (1998). The checkpoint protein MAD2 and the mitotic regulator CDC20 form a ternary complex with the anaphase-promoting complex to control anaphase initiation. Genes Dev 12, 1871-1883;
12. Tang, Z., Bharadwaj, R., Li, B. and Yu, H. (2001). Mad2-Independent inhibition of APCCdc20 by the mitotic checkpoint protein BubR1. Dev Cell 1, 227-237.
13. Fang, G. (2062). Checkpoint protein BubR1 acts synergistically with Mad2 to inhibit anaphase-promoting complex. Mol Biol Cell 13, 755-766.
14. Sudakin, V., Chan, G. K. and Yen, T. J. (2001). Checkpoint inhibition of the APC/C in HeLa cells is mediated by a complex of BUBR1, BUB3, CDC20, and MAD2. J Cell Biol 154, 925-936.
15. Li, Y., Gorbea, C., Mahaffey, D., Rechsteiner, M. and Benezra, R. (1997). MAD2 associates with the cyclosome/anaphase-promoting complex and inhibits its activity. Proc Natl Acad Sci USA 94, 12431-12436.
16. Skoufias, D. A., Andreassen, P. R., Lacroix, F. B., Wilson, L. and Margolis, R. L. (2001). Mammalian mad2 and bub1/bubR1 recognize distinct spindle-attachment and kinetochore-tension checkpoints. Proc Natl Acad Sci USA 98, 4492-4497.
17. Cohen, J. (2002). Sorting out chromosome errors. Science 296, 2164-2166.
18. Lengauer, C., Kinzler, K. W. and Vogelstein, B. (1998). Genetic instabilities in human cancers. Nature 396, 643-649.
19. Nakagawa, H., Yokozaki, H., Oue, N., Sugiyama, M., Ishikawa, T., Tahara, E. and Yasui, W. (2002). No mutations of the Bub 1 gene in human gastric and oral cancer cell lines. Oncol Rep 9, 1229-1232.
20. Shigeishi, H., Yokozaki, H., Kuniyasu, H., Nakagawa, H., Ishikawa, T., Tahara, E. and Yasui, W. (2001). No mutations of the Bub1 gene in human gastric carcinomas. Oncol Rep 8, 791-794.
21. Reis, R. M., Nakamura, M., Masuoka, J., Watanabe, T., Colella, S., Yonekawa, Y., Kleihues, P. and Ohgaki, H. (2001). Mutation analysis of hBUB1 1, hBUBR1 and hBUB3 genes in glioblastomas. Acta Neuropathol (Ber1) 101, 297-304.
22. Sato, M., Sekido, Y., Horio, Y., Takahashi, M., Saito, H., Minna, J. D., Shimokata, K. and Hasegawa, Y. (2000). Infrequent mutation of the hBUB1 and hBUBR1 genes in human lung cancer. Jpn J Cancer Res 91, 504-509.
23. Myrie, K. A., Percy, M. J., Azim, J. N., Neeley, C. K. and Petty, E. M. (2000). Mutation and expression analysis of human BUB1 and BUB1B in aneuploid breast cancer cell lines. Cancer Lett 152, 193-199.
24. Cahill, D. P., Lengauer, C., Yu, J., Riggins, G. J., Willson, J. K., Markowitz, S. D., Kinzler, K. W. and Vogelstein, B. (1998). Mutations of mitotic checkpoint genes in human cancers. Nature 392, 300-303.
25. Tighe, A., Johnson, V. L., Albertella, M. and Taylor, S. S. (2001). Aneuploid colon cancer cells have a robust spindle checkpoint. EMBO Rep 2, 609-614.
26. Michel, M. L., Liberal, V., Chatterjee, A., Kirchwegger, R., Pasche, B., Gerald, W., Dobles, M., Sorger, P. K., Murty, V. V. V. S. and Benezra, R. (2001). MAD2 haploinsufficiency causes premature anaphase and chromosome instability in mammalian cells. Nature 409, 355-359.
27. Babu, J. R., Jeganathan, K. B., Baker, D. J., Wu, X., Kang-Decker, N. and Van Deursen, J. M. (2003). Rael is an essential mitotic checkpoint regulator that cooperates with Bub3 to prevent chromosome missegregation. J Cell Biol 160, 341-353.
28. Kalitsis, P., Earle, E., Fowler, K. J. and Choo, K. H. (2000). Bub3 gene disruption in mice reveals essential mitotic spindle checkpoint function during early embryogenesis. Genes Dev 14, 2277-2282.
29. Putkey, F. R., Cramer, T., Morphew, M. K., Silk, A. D., Johnson, R. S., McIntosh, J. R. and Cleveland, D. W. (2002). Unstable kinetochore-rnicrotubule capture and chromosomal instability following deletion of CENP-E. Dev Cell 3, 351-365.
30. Dobles, M., Liberal, V., Scott, M. L., Benezra, R. and Sorger, P. K. (2000). Chromosome missegregation and apoptosis in mice lacking the mitotic checkpoint protein Mad2. Cell 101, 635-645.
31. Brummelkamp, T. R., Bemards, R. and Agami, R. (2002). A system for stable expression of short interfering RNAs in mammalian cells. Science 296, 550-553.
32. Taylor, S. S., Ha, E. and McKeon, F. (1998). The human homologue of Bub3 is required for kinetochore localization of Bub1 and a Mad3/Bub1-related protein kinase. J Cell Biol 142, 1-11.
33. Mao, Y., Abrieu, A. and Cleveland, D. W. (2003). Activating and silencing the mitotic checkpoint through CENP-E-dependent activation/inactivation of BubR1. Cell 114, 87-98.
34. Rieder, C. L., Schultz, A., Cole, R. and Sluder, G. (1994). Anaphase onset in vertebrate somatic cells is controlled by a checkpoint that monitors sister kinetochore attachment to the spindle. J Cell Biol 127, 1301-1310.
35. Hagting, A., Den Elzen, N., Vodermaier, H. C., Waizenegger, I. C., Peters, J. M. and Pines, J. (2002). Human securin proteolysis is controlled by the spindle checkpoint and reveals when the APC/C switches from activation by Cdc20 to Cdh1. J Cell Biol 157, 1125-1137.
36. Waizenegger, I. C., Hauf, S., Meinke, A. and Peters, J. M. (2000). Two distinct pathways remove mammalian cohesin from chromosome arms in prophase and from centromeres in anaphase. Cell 103, 399-410.
37. Wu, H., Lan, Z., Li, W., Wu, S., Weinstein, J., Sakamoto, K. M. and Dai, W. (2000). p55CDC/hCDC20 is associated with BUBR1 and may be a downstream target of the spindle checkpoint kinase. Oncogene 19, 4557-4562.
38. Kaplan, K. B., Burds, A. A., Swedlow, J. R., Bekir, S. S., Sorger, P. K. and Nathke, I. S. (2001). A role for the Adenomatous Polyposis Coli protein in chromosome segregation. Nat Cell Biol 3, 429-432.
39. Jordan, M. A., Wendell, K., Gardiner, S., Derry, W. B., Copp, H. and Wilson, L. (1996). Mitotic block induced in HeLa cells by low concentrations of paclitaxel (Taxol) results in abnormal mitotic exit and apoptotic cell death. Cancer Res 56, 816-825.
40. Jordan, M. A., Toso, R. J., Thrower, D. and Wilson, L. (1993). Mechanism of mitotic block and inhibition of cell proliferation by taxol at low concentrations. Proc Natl Acad Sci USA 90, 9552-9556.
41. Blagosklonny, M. V. and Fojo, T. (1999). Molecular effects of paclitaxel: myths and reality (a critical review). Int J Cancer 83, 151-156.
42. Haldar, S., Chintapalli, J. and Croce, C. M. (1996). Taxol induces bcl-2 phosphorylation and death of prostate cancer cells. Cancer Res 56, 1253-1255.
43. Scatena, C. D., Stewart, Z. A., Mays, D., Tang, L. J., Keefer, C. J., Leach, S. D. and Pietenpol, J. A. (1998). Mitotic phosphorylation of Bcl-2 during normal cell cycle progression and Taxol-induced growth arrest. J Biol Chem 273, 30777-30784.
44. Hoffman, D. B., Pearson, C. G., Yen, T. J., Howell, B. J. and Salmon, E. D. (2001). Microtubule-dependent changes in assembly of rmicrotubule motor proteins and mitotic spindle checkpoint proteins at PtKl kinetochores. Mol Biol Cell 12, 1995-2009.
45. Medema, R. H., Kops, G. J., Bos, J. L. and Burgering, B. M. (2000). AFX-like Forkhead transcription factors mediate cell-cycle regulation by Ras and PKB through p27kip1. Nature 404, 782-787.
46. Brown, K. D., Wood, K. W. and Cleveland, D. W. (1996). The kinesin-like protein CENP-E is kinetochore-associated throughout poleward chromosome segregation during anaphase-A. J Cell Sci 109, 961-969.
47. Lee, M. K., Xu, Z., Wong, P. C. and Cleveland, D. W. (1993). Neurofilaments are obligate heteropolymers in vivo. J Cell Biol 122, 1337-1350.

Each of the foregoing patents, patent applications and references is hereby incorporated by reference.

While the invention has been described with respect to certain embodiments, it should be appreciated that many modifications and changes may be made by those of ordinary skill in the art without departing from the spirit of the invention. It is intended that such modification, changes and equivalents fall within the scope of the following claims.

What is claimed is:

1. A method for inducing apoptosis in a cell comprising
reducing expression or activity of one or more mitotic checkpoint molecules.

2. The method of claim 1, wherein the expression of the one or more mitotic checkpoint molecules is reduced by contacting the cell with a siRNA specific for the one or more mitotic checkpoint molecules.
preferably wherein the mitotic checkpoint molecule is BubR1, Mad2, Bub3 or CENP-E.

3.-6. (canceled)

7. The method of claim 1, wherein the activity of the one or more mitotic checkpoint molecules is reduced by contacting the cell with an antibody that binds to the mitotic checkpoint molecule,
optionally wherein the antibody is selected from the group consisting of monoclonal antibodies, human antibodies, humanized antibodies, chimerized antibodies, and antigen-binding fragments thereof,
preferably wherein the mitotic checkpoint molecule is BubR1, Mad2, Bub3 or CENP-E.

8.-12. (canceled)

13. The method of claim 1, wherein activity is reduced by contacting the cell with a molecule that inhibits kinase activity of the one or more mitotic checkpoint molecules,
preferably wherein the mitotic checkpoint molecule is BubR1.

14. (canceled)

15. A method for treating cancer or a hyperproliferative cell disease comprising:
administering to a subject in need of such treatment an effective amount of an agent that reduces expression or activity of one or more mitotic checkpoint molecules.

16. The method of claim 15, wherein the expression of the one or more mitotic checkpoint molecules is reduced by administering a siRNA specific for the one or more mitotic checkpoint molecules, preferably wherein the mitotic checkpoint molecule is BubR1, Mad2, Bub3 or CENP-E.

17.-20. (canceled)

21. The method of claim 15, wherein the activity of the one or more mitotic checkpoint molecules is reduced by administering an antibody that binds to the mitotic checkpoint molecule,
optionally wherein the antibody is selected from the group consisting of monoclonal antibodies, human antibodies, humanized antibodies, chimerized antibodies, and antigen-binding fragments thereof,
preferably wherein the mitotic checkpoint molecule is BubR1, Mad2, Bub3 or CENP-E.

22.-26. (canceled)

27. The method of claim 15, wherein activity is reduced by administering a molecule that inhibits kinase activity of the one or more mitotic checkpoint molecules,
preferably wherein the mitotic checkpoint molecule is BubR1.

28. (canceled)

29. The method of claim 15 wherein an anti-cancer therapy is used in combination with the agent,
preferably wherein the anti-cancer therapy is chemotherapy,
optionally wherein the chemotherapy is one or more microtubule poison drugs, and wherein the chemotherapy is not co-administered with the agent.

30.-45. (canceled)

46. A composition comprising a therapeutically effective amount of a siRNA specific for a mitotic checkpoint molecule,
preferably wherein the mitotic checkpoint molecule is BubR1, Mad2, Bub3 or CENP-E.

47.-53. (canceled)

54. The composition of claim 46, further comprising a pharmaceutically acceptable carrier.
